(19) United States
(12) Patent Application Publication
(10) Pub. No.: US 2010/0047767 A1
(43) Pub. Date: Feb. 25, 2010

(54) PATHOGEN BINDING

(75) Inventors: Prabhjyot Dehal, Dundee (GB); David Pritchard, Dundee (GB); Claire Geekie, Dundee (GB)

Correspondence Address: Joseph R. Baker, APC;Gavrilovich, Dodd & Lindsey LLP, 4660 La Jolla Village Drive, Suite 750, San Diego, CA, 92122, US

(73) Assignee: ITI SCOTLAND LIMITED, Glasgow (GB)

(21) Appl. No.: 12/524,966

(22) PCT Filed: Feb. 4, 2008
(86) PCT No.: PCT/EP08/51361

§ 371 Date: Oct. 27, 2009

(30) Foreign Application Priority Data
Feb. 5, 2007 (GB) 0702183.5

(51) Int. Cl. C12Q 1/70; G01N 33/53; G01N 33/554; C07K 14/00

(52) U.S. Cl. 435/5; 435/7.2; 435/7.32; 530/402

(57) ABSTRACT

Provided is a method for determining the presence or absence of a pathogen in a sample, which method comprises: a) contacting the sample with a whole or a part of a cell surface receptor protein capable of binding the pathogen; b) allowing the cell surface receptor protein or part thereof to bind the pathogen; c) determining the presence or absence of the pathogen bound to the receptor protein or part thereof.

The present invention concerns a method for detecting a pathogen in a sample. The method comprises contacting the sample with a whole or a part of a cell surface receptor protein capable of binding the pathogen. Preferably, the cell surface receptor protein is one which binds the pathogen during a wild-type infection of the pathogen in vivo. The invention also concerns methods for diagnosis of a subject, and fusion proteins for use in the methods.

Increasing the sensitivity of detection of pathogens for improved diagnostics is an important goal. Conventional assays for pathogen detection in samples taken from subjects or patients have their limitations. The most commonly used means of achieving, for example, detection of hepatitis C virus (HCV) has been to amplify minute amounts of nucleic acids or use antibodies to detect viral proteins (Erensoy (2001). Diagnosis of hepatitis C virus (HCV) infection and laboratory monitoring of its therapy. J Clin Virol. 21 (3) 271-81; Kuo et al. (1989). An assay for circulating antibodies to a major etiologic virus of human non-A, non-B hepatitis. Science 244 362-4; Hodinka, R. L. (1998). The clinical utility of viral quantitation using molecular methods. Clinical and Diagnostic Virology. 10 25-47; Kurtz, et al. (2001). The diagnostic significance of an assay for ‘total’ hepatitis C core antigen. Journal of Virological Methods. 96 (2) 127-32; Trimoulet, et al. (2002). Evaluation of the VERSANT HCV RNA 3.0 Assay for Quantification of Hepatitis C Virus RNA in Serum. Journal of Clinical Microbiology. 40 (6) 2031-2036; Strader et al. (2004). Diagnosis, management and treatment of hepatitis C. Hepatology 39 (4) 1147-71). These methods are suitable where relatively large amounts of a patient samples are available (for example, several millilitres of blood), and enough nucleic acids or antigens are available for detection. However, detection of low titre virus in smaller volumes of blood, and the ability to concentrate and purify pathogens from a sample would greatly aid diagnosis.

One solution is provided by Grinde B et al. (1995). Sensitive detection of group A rotaviruses by immunomagnetic separation and reverse transcription-polymerase chain reaction. J. Virol. Methods. 55 (3) 327-38, which demonstrates the use of magnetic beads coated with monoclonal antibodies directed against the group-specific, inner capsid protein (VP6) of group A rotaviruses. Once the virus is captured and purified with the help of a magnet, the genome was made available for reverse transcription by heat-disruption of the viral particles, thus enabling both detection and analysis of the virus.

An obvious method of concentrating virus would be to use antibody coated inert media. However, such a solution has its own problems. For example in the detection of an RNA virus, such as HCV, the genome of the virus is subject to continual mutation and epitopes on the surface of the virus may “escape” detection by high affinity antibodies. In fact, it has been shown that an individual patient does not have one singular form of HCV. Rather, the virus exists as a “quasi-species” with many different genomes, with therefore slightly different surface proteins within its host. With such viruses it can be difficult to obtain a correct analysis of infection.

As a result there is a need for an improved method by which the presence or absence of pathogens in a sample can be determined and diagnosed.

Accordingly, the present invention provides a method for determining the presence or absence of a pathogen in a sample, which method comprises:

a) contacting the sample with a whole or a part of a cell surface receptor protein capable of binding the pathogen.
b) allowing the cell surface receptor protein or part thereof to bind the pathogen;
c) determining the presence or absence of the pathogen bound to the receptor protein or part thereof.

The invention also provides a method for capturing, concentrating, purifying and/or isolating a pathogen in a sample, which method comprises:

a) contacting the sample with a whole or a part of a cell surface receptor protein capable of binding the pathogen;
b) allowing the cell surface receptor protein or part thereof to bind the pathogen;
c) determining the presence or absence of the pathogen bound to the receptor protein or part thereof.

The methods of the present invention may be applied to any system, but are especially suited to microfluidic or nanofluidic systems, and in particular to a diagnostic flow process taking place in such a system.

It is also especially preferred that the method comprises contacting the sample with only that part of the cell surface receptor protein which binds to the pathogen (e.g. the large extracellular loop (LEL) in CD81). This has the advantage of simplifying the means used to bind to the pathogen, whilst also reducing the size of the molecular species involved, which is particularly suitable for microfluidic and nanofluidic systems. Previously, the reliability of methods which employ only the parts of the receptor that take part in binding pathogens has been problematic. However, the present invention has overcome these problems and a reliable process has been developed.

In a preferred aspect the method further comprises the step of concentrating the bound pathogen.

This method is particularly advantageous because it allows the concentration of low titre pathogen from a sample before detection. As a result pathogens can be detected in relatively small samples, and the sensitivity of current assays can be increased. Accordingly, those patients with relatively low levels of pathogens can be identified.

In particular, the method of the present invention can be used to concentrate and detect intact hepatitis C virus from blood or other tissue using the binding affinity of the virus for CD81, CD209, or CD209L receptors, or fragments thereof. These proteins or fragments thereof can be generated conjugated to inert surfaces. Coupling of these receptors to tags (for example, as a CD81-GST fusion protein) either on their own or as mixtures onto surfaces, such as magnetic or other beads (such as agarose), enables the separation and concentration of virus from the blood or tissue matrix. The attached virus can then be disrupted for detection and downstream analysis of proteins, nucleic acids and lipids.

The CD81 receptor has been shown to bind the E1E2 surface complex of HCV particles with high affinity, and is essential for viral entry into cells. The different genotypes of HCV all bind CD81 with slightly different affinities. If the viral amino acid residues which are critical for binding were to no longer able to effectively dock due to mutation, the virus would become non-viable, and would not be able to enter cells and replicate. Accordingly, the advantage of the present invention is that only infectious viral particles, i.e. only those capable of binding to the CD81 receptor, will be detected.

The present invention will be described further by way of example only, with reference to the following Figures:

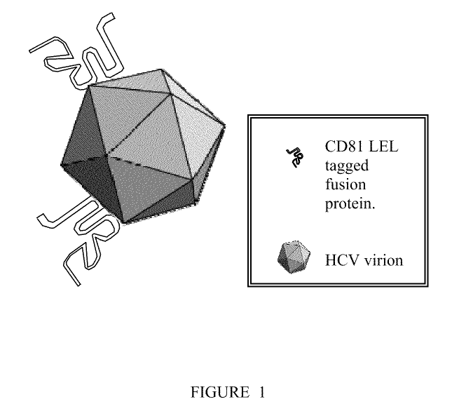

FIG. 1 shows a schematic of a hepatitis C virion bound to a CD81 tagged fusion protein.

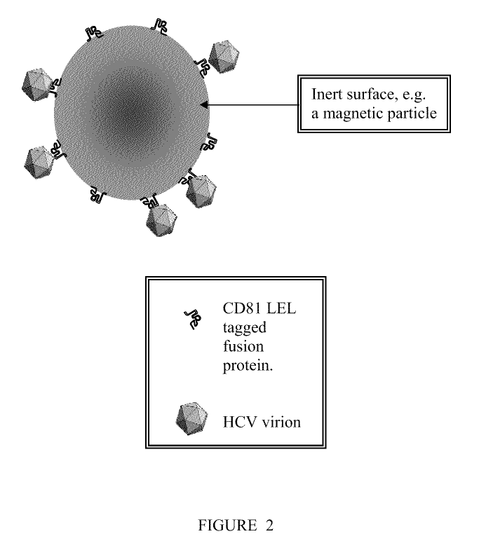

FIG. 2 shows a schematic of hepatitis C virions bound to a magnetic particle via CD81 LEL tagged fusion proteins attached to the surface of the magnetic particle.

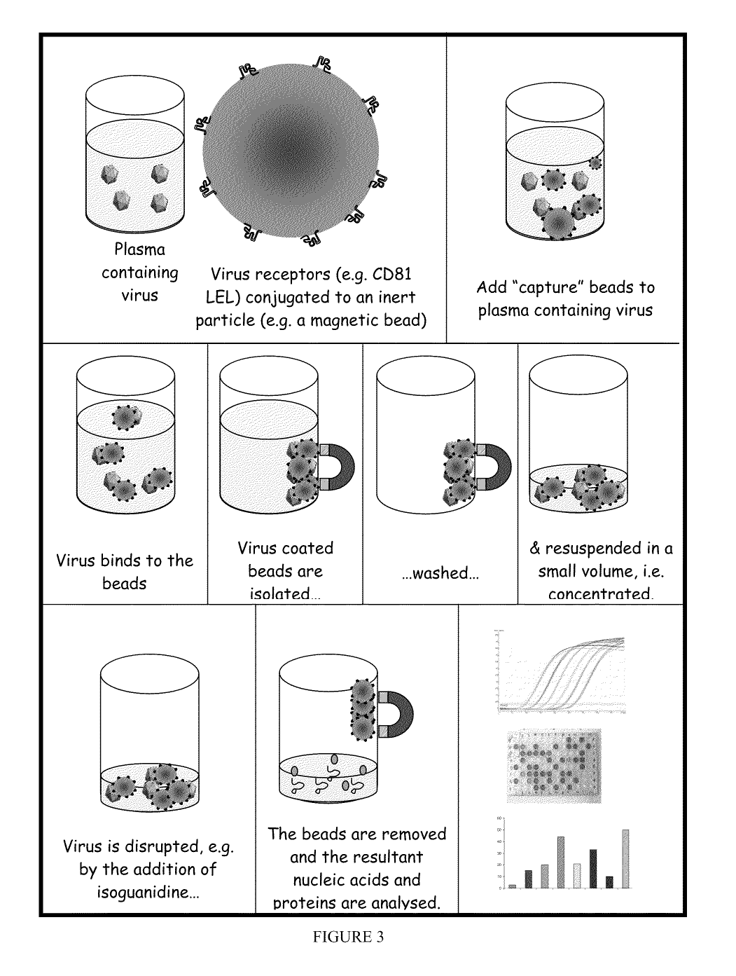

FIG. 3 is a schematic showing an embodiment of the method of the present invention in which virus from a plasma sample is concentrated, detected and analysed.

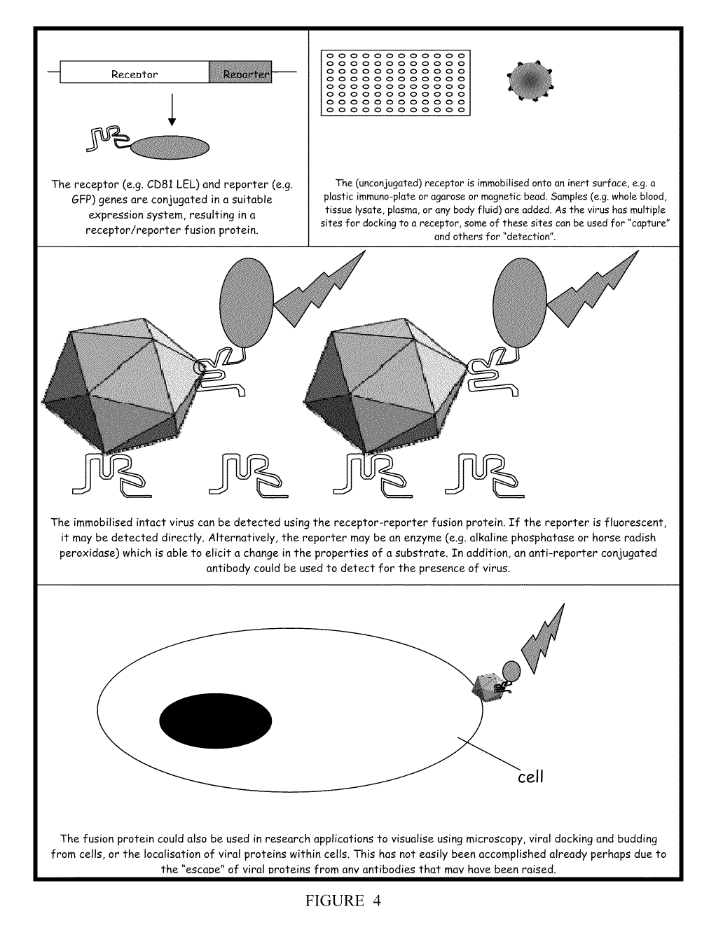

FIG. 4 is a schematic showing an embodiment of the method of the present invention wherein the report GFP protein is used to detect the presence or absence of virus in a sample.

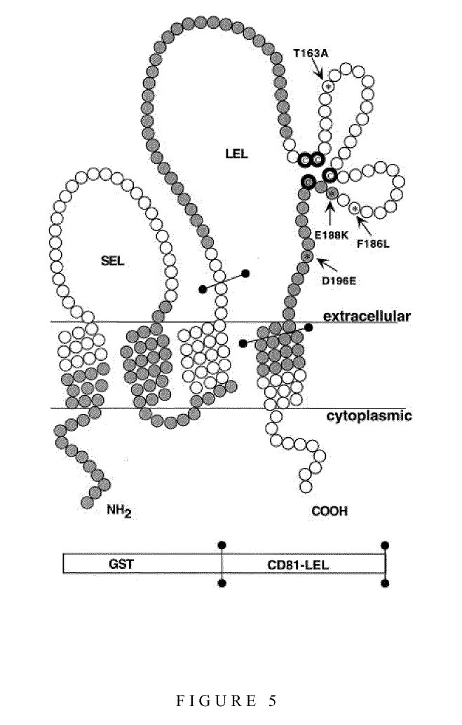

FIG. 5 shows the structure of CD81 in situ in a cellular membrane. The LEL can be seen.

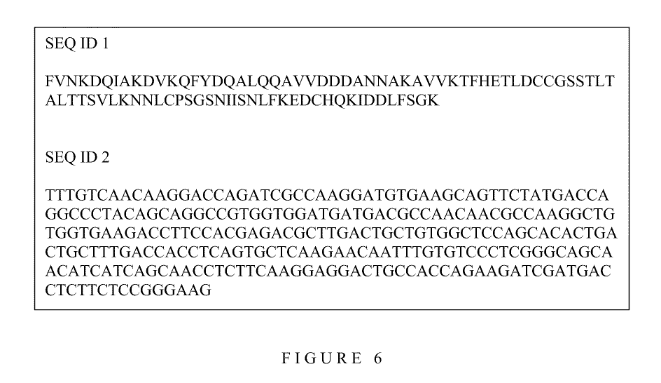

FIG. 6 shows the amino acid sequence (SEQ ID 1) and DNA sequence (SEQ ID 2) of the CD81LEL used in Example 1.

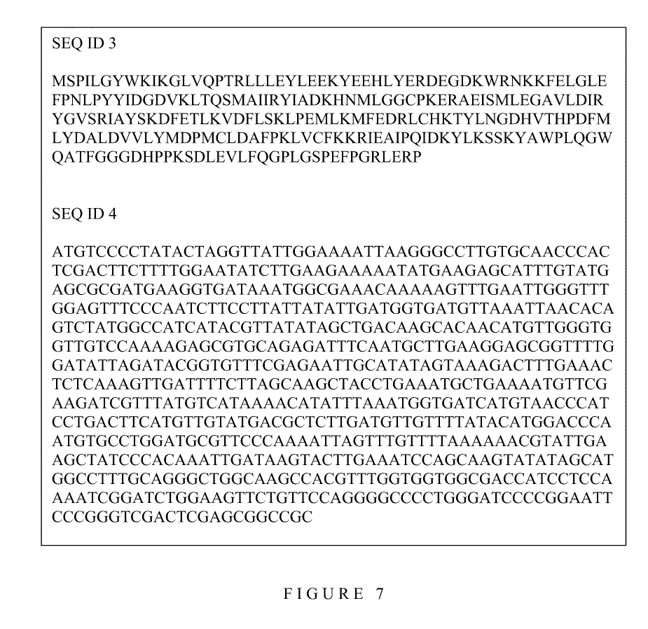

FIG. 7 shows the amino acid sequence (SEQ ID 3) and DNA sequence (SEQ ID 4) of the GST gene from the pGEX6p-1 vector.

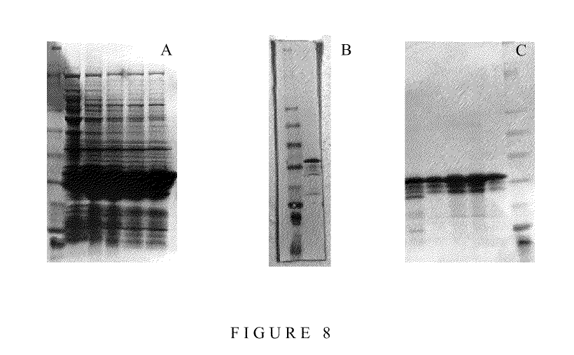

FIG. 8 shows: (A) an SDS-PAGE gel of the inclusion bodies isolated from the bacterial cell culture expressing the CD81LEL-GST fusion protein; (B) a Western blot showing a high molecular weight band likely to be CD81LEL-GST (diluted×16)—the fainter bands correspond to breakdown products and GST; (C) elution fractions from a glutathione column, lane 1: initial inclusion body prep, lanes 2-5 elution fractions.

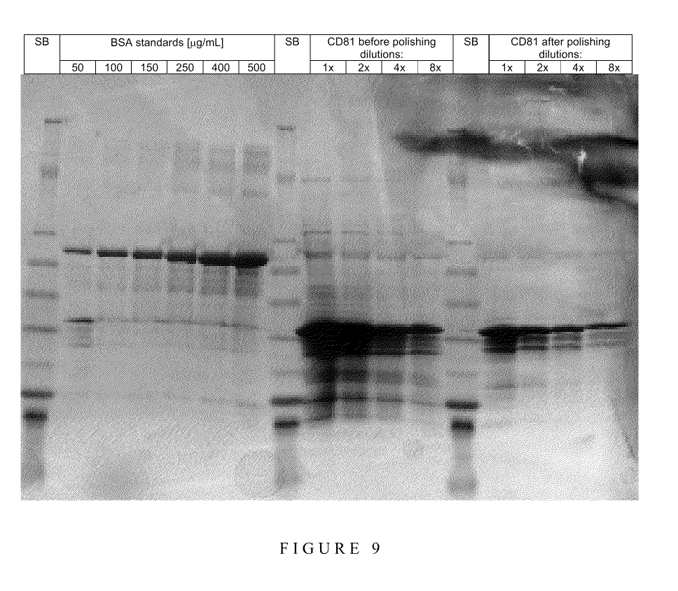

FIG. 9 shows protein concentration determination using comparison to known BSA quantities.

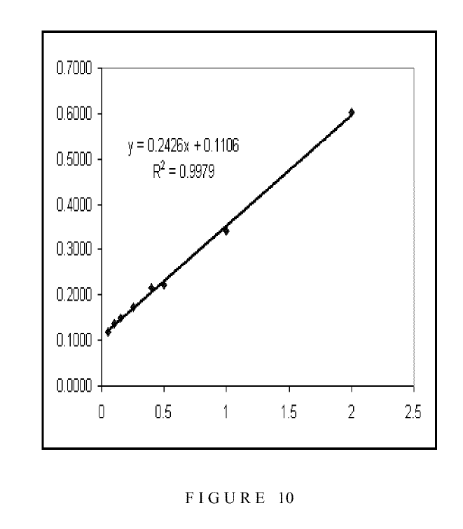

FIG. 10 shows protein concentration determination using a protein assay kit (BSA standard curve).

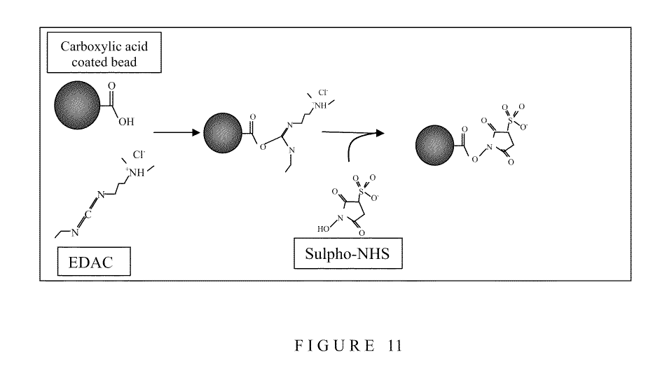

FIG. 11 shows the chemistries involved in binding proteins to carboxyl groups.

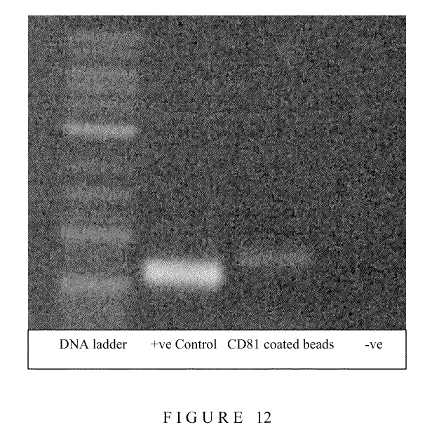

FIG. 12 shows the gel electrophoresis of PCR amplified eGFP gene from HCVpp. Key: +ve control, HCVpp mixed directly with RNA isolation beads, CD81 coated beads: HCVpp concentrated using CD81LEL-GST covalently coupled to magnetic beads, −ve: negative control (no cDNA template).

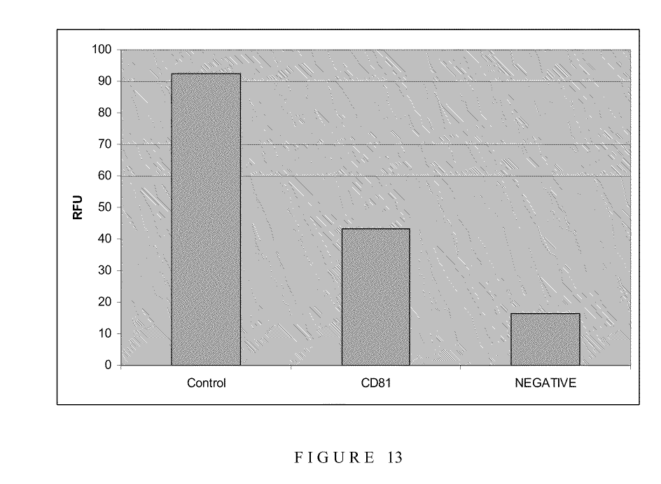

FIG. 13 shows fluorescence measurements of the three PCR products. Excitation wavelength: 497 nm, emission reading at 520 nm n.

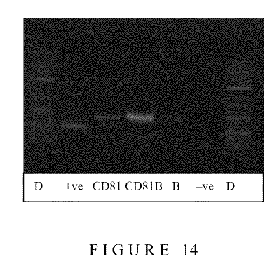

FIG. 14 shows gel electrophoresis of PCR amplified eGFP gene from HCVpp. Key: D: 2 log-DNA ladder, +ve: positive control, HCVpp mixed directly with RNA isolation beads, CD81: HCVpp concentrated using CD81LEL-GST covalently coupled to magnetic beads, CD81B HCVpp concentrated using biotinylated CD81LEL-GST coupled to streptavidin coated magnetic beads −ve: negative control (no cDNA template).

The present invention relates to a method for determining the presence or absence of a pathogen in a sample, which method comprises:

a) contacting the sample with a whole or a part of a cell surface receptor protein capable of binding the pathogen:
b) allowing the cell surface receptor protein or part thereof to bind the pathogen;
c) determining the presence or absence of the pathogen bound to the receptor protein or part thereof.

The pathogen of the present invention is not particularly limited and may be any pathogen which binds to a specific cell surface receptor during a wild-type infection in vivo. The pathogen may be selected from DNA and RNA viruses, bacteria, fungi, parasites, and prions. The method of the present invention is particularly useful with RNA viruses which, as described above, have RNA genomes that are subject to continual mutation such that the epitopes on the surface of the virus may vary.

The method of the present invention can be used to determine the presence or absence of a pathogen in a sample taken from a subject or in an environmental sample. Therefore, the term “sample” is not especially limiting. Where the sample is taken from a subject, it may be a body fluid such as whole blood, urine, plasma, spinal fluid or serum, or it may be a crude lysate of solid tissue or cells. Alternatively, the sample may be from a tissue culture. Where the sample is taken from the environment, it may be a soil sample, an air sample or a fluid sample, such as a water sample.

The sample may be subjected to processing steps before it is used in the present method. For example, a sample may be cultivated in vitro before being used in the present invention.

In a preferred aspect of the present invention the pathogen is the hepatitis C virus, and the method of the present invention can be used to detect intact hepatitis C virions from blood or other tissues. In a particularly preferred aspect the method of the present invention can be used to concentrate and detect intact hepatitis C virions from blood or other tissues.

The nature of the cell surface receptor, or part thereof, is not especially limited, provided it binds specifically to the pathogen. Preferably the receptor, or part thereof, is one which binds to the pathogen during a wild-type infection of the pathogen in vivo. In particular, this wild-type infection is an infection of a mammal, for example a human.

The term “part thereof” refers to a fragment of the cell surface receptor protein. Preferably the fragment must be sufficiently long for it to be able to form the correct conformational shape (i.e. the wild-type shape) in order to allow binding to the virus. However, shorter fragments may also be used as long as they bind the virus with high enough affinity to allow the virus to be efficiently detected and/or separated. In particular in one embodiment of this invention, the fragment preferably includes the part of the receptor which is exposed on the outer membrane of the cell surface in vivo.

Preferably the receptor or part thereof is one which binds the pathogen with high affinity or avidity. In particular, the receptor is one which binds the pathogen with a K_{D} (dissociation constant) of 10⁻⁴ M or better. Most preferably, the receptor is one which binds the pathogen with a K_{D} of 10⁻⁶ M. These values are comparable to those used successfully in the methods of the prior art. For example, antibodies have been used which have a K_{D} of 10⁻⁴ M. Further, glutathione and glutathione S-transferase have a K_{D} (dissociation constant) of around 10⁻⁶ M, which is considered “good”, and the GST tag has been used to purify recombinant proteins (Nieslanik and Atkins, (2000), The Catalytic Tyr-9 of Glutathione S-Transferase A1-1 Controls the Dynamics of the C terminus. J. Biol. Chem. 275 (23) 17447-51). Natural antibodies have an affinity ceiling of around 10⁻⁹ M. Further, CD81 has been shown to bind HCV proteins with a K_{D} of around 10⁻⁹ M too, which is considered “strong”. Biotin binds streptavidin with a K_{D} of 10⁻¹⁵ M and is considered “very strong-almost covalent” (Boder et al., (2000), “Directed evolution of antibody fragments with monovalent femtomolar (10⁻¹⁵ M) antigen-binding affinity.” PNAS. 97 (20) 10701-5).

In a preferred embodiment the cell surface receptor is one which during a wild-type infection in vivo allows, or is in part responsible for, the pathogen binding and/or entering the cell.

In a further preferred embodiment the cell surface receptor protein or part thereof binds the pathogen to form a protein-pathogen complex.

The whole or a part of the cell surface receptor protein can be used in the method of the present invention. In one aspect of the present invention the protein, or part thereof, is used as a fusion protein, or coupled to a tag. Suitable tags or fusion partners include glutathione-S-transferase (GST), which has been shown to dimerise and enhance binding, and green fluorescent protein (GFP).

In particular, the GST tag can be used in methods where concentration, separation, or purification of the pathogen particles is required. The GST tag is fused to the N- or C-terminus of the cell surface receptor protein, or part thereof. The GST-fusion protein can be easily produced in bacterial systems, such as E. coli, as a recombinant protein. After contacting the GST-fusion protein with the sample, and allowing it to bind the pathogen, the GST-fusion protein-pathogen complex may be separated from the sample by contacting it with the GST substrate, glutathione. In particular, this glutathione may be coated on sepharose beads. Once the complex has bound to the beads they may be washed, to remove the rest of the sample, thus separating and concentrating the pathogen from the rest of the sample.

In another embodiment of the present invention the cell surface receptor protein, or part thereof, is used as a fusion protein with GFP or other reporter protein. Such a fusion protein may be used in a manner similar to an antibody, to detect the presence or absence of a pathogen in a sample. The schematic provided by FIG. 4 demonstrates how this embodiment works. Specifically, the receptor (e.g. CD81 LEL) and reporter (e.g. GFP) genes can be cloned into a plasmid containing suitable promoter and polyadenylation sequences, and also a purification tag if required (e.g. GST, polyhistidine, IgGFc, etc.). The genes run concurrently and may be separated by a small linker region. The plasmid is then transduced or transfected into cells (bacterial or eukaryotic). The resultant expressed recombinant receptor/reporter fusion protein can be purified using established techniques (e.g. glutathione columns for the GST tag, anti receptor or reporter antibody coated columns).

The (unconjugated) receptor is immobilised onto an inert surface, e.g. a plastic immuno-plate or agarose or magnetic bead. The magnetic beads are preferably 10 nm or more in diameter, more preferably 10 nm to 10 μm in diameter, still more preferably 100 nm to 5 μm in diameter. Examples of commercial sources of these beads are Dynabeads (Invitrogen), MACS beads (Miltenyi Biotec), Bio-Adembeads (Ademtech). Suitable agarose beads are also commercially available and can be obtained, for example, from Polysciences, Inc.

The immobilised intact pathogen can be detected using the receptor/reporter fusion protein. If the reporter is fluorescent, it may be detected directly. Alternatively, the reporter may be an enzyme (e.g. alkaline phosphatase or horse radish peroxidase) which is able to elicit a change in the chemical properties of a substrate. In addition, an anti-reporter conjugated antibody could be used to detect for the presence or absence of pathogen.

Where the pathogen is a virus which has multiple sites for docking to a receptor, some of these sites can be used for capture and others for detection.

The fusion proteins may be coupled onto an inert or solid surface. Suitable surfaces include magnetic or agarose beads, to which the fusion protein could be coupled using a number of available coupling chemistries, such as passive adsorption (e.g. using sodium bicarbonate buffer), ionic adsorption, cyanogen bromide, carbodiimide, nickel/histidine, photochemical reactions. Alternatively, the fusion protein may comprise a further protein or peptide, which may enable it to be coupled to the surface. For example, a biotin tag may be added to the fusion protein for coupling it to avidin coated beads.

Potential RNA viruses and their respective cell surface receptors are: hepatitis C virus and CD81 (in particular the large extracellular loop (LEL) of CD81; hepatitis C virus and CD209; hepatitis C virus and CD209L; rhinovirus and ICAM1; human immunodeficiency virus and CD4; human immunodeficiency virus and CCR5; influenza and sialoglycoproteins.

Potential DNA viruses and their respective cell surface receptors are: herpes simplex virus and nectin-1 (Spear et al. (2006). Different receptors binding to distinct interfaces on herpes simplex virus gD can trigger events leading to cell fusion and viral entry. Virology. 344 (1) 17-24; Compton (2004). Receptors and immune sensors: the complex entry path of human cytomegalovirus. Trends in Cell Biology. 14 (1)); Cytomegalovirus (CMV) (also a herpes virus) and epidermal growth factor receptor (EGFR) (Spear. (2004). Herpes simplex virus: receptors and ligands for cell entry. Cell Microbiol. 6 (5):401-10); measles and CD150 (Sidorenko and Clark, (2003). The dual-function CD150 receptor subfamily: the viral attraction. Nat Immunol. 4 (1) 19-24); Epstein-Barr virus (which causes glandular fever and a number of cancers) and complement receptor 2 (CR2) (Fingeroth et al. (1984). Epstein-Barr virus receptor of human B lymphocytes is the C3d receptor CR2. PNAS. 81 4510-14).

In one aspect of the invention the pathogen is hepatitis C virus (HCV) and the cell surface receptor is CD81. HCV is a positive strand RNA virus of the flaviviridae family. CD81, a cell surface receptor expressed on various cell types including hepatocytes and B lymphocytes has been shown to bind the HCV envelope protein E2. In particular, the large extracellular loop (LEL) of CD81, has been shown to bind the HCV particle (Pileri et al., (1998). Binding of Hepatitis C virus to CD81. Science. 282 938-41). In addition, other receptors of use include CD209 and CD209L (Cormier et al., (2004). L-SIGN(CD209L) and DC-SIGN (CD209) mediate transinfection of liver cells by hepatitis C virus. PNAS 101 (39) 14067-14072) or peptides derived from these.

These and other virus-receptor pairings are shown below in Table 1.

TABLE 1
Suitable viruses and receptor pairings
Virus: receptor

Adenoviruses: Integrins/CAR/HS/GAGs/CD46/CD80/CD86
Arboviruses: various receptors including insect receptors
Astrovirus: CD155
hCMV: EGFR (a herpes virus)
Dengue virus: DC-SIGN
Ebola virus: Folate receptor-α, DC-SIGN
Epstein Barr: CR2 (CD21)
FIV: CD9
Foot and Mouth: Integrins (specifically αvβ3, αvβ6 and αvβ1)
Herpes simplex: TNF family and nectin-1 and IgG
HIV: CD4
HTLV: GLUT1 glucose transporter and Neuropilin-1
Influenza A virus (IV): sialic acid
Measles: SLAM (CD150), CD46
Nipah virus: EphrinB2 receptor.
Norwalk virus: H type 2 histo-blood group antigen
Human papilloma virus 16: syndecan-1
Human parvovirus B19: P antigen
Picornavirus
Polio virus: CD155
Rabies virus: nAChR, NCAM, p75NTR
Reovirus: JAM-1
human rhinovirus (HRV): ICAM-1
Rotavirus: Integrins (α2β1, αvβ3 and hsc90)
SARS-CoV: ACE2
Semliki Forest virus (SFV):

Suitable bacteria and receptor pairings are shown in Table 2 below.

TABLE 2
Suitable bacteria and receptor pairings

| Bacterium | Adhesin | Receptor | Attachment site | Disease |
|---|---|---|---|---|
| Streptococcus | Protein F | Amino terminus of | Pharyngeal | Sore throat |
| pyogenes | | fibronectin | epithelium | |
| Streptococcus | Glycosyl transferase | Salivary | Pellicle of tooth | Dental caries |
| mutans | | glycoprotein | | |
| Streptococcus | Lipoteichoic acid | Unknown | Buccal | None |
| salivarius | | | epithelium of | |
| | | | tongue | |
| Streptococcus | Cell-bound protein | N- | Mucosal | pneumonia |
| pneumoniae | | acetylhexosamine- | epithelium | |
| | | galactose | | |
| | | disaccharide | | |
| Staphylococcus | Cell-bound protein | Amino terminus of | Mucosal | Various |
| aureus | | fibronectin | epithelium | |
| Neisseria | N-methylphenyl- | Glucosamine- | Urethral/cervical | Gonorrhea |
| gonorrhoeae | alanine pili | galactose | epithelium | |
| | | carbohydrate | | |
| Enterotoxigenic | Type-1 fimbriae | Species-specific | Intestinal | Diarrhea |
| E. coli | | carbohydrate(s) | epithelium | |
| Uropathogenic | Type 1 fimbriae | Complex | Urethral | Urethritis |
| E. coli | | carbohydrate | epithelium | |
| Uropathogenic | P-pili (pap) | Globobiose linked | Upper urinary | Pyelonephritis |
| E. coli | | to ceramide lipid | tract | |
| Bordetella | Fimbriae | Galactose on | Respiratory | Whooping |
| pertussis | (“filamentous | sulfated glycolipids | epithelium | cough |
| | hemagglutinin”) | | | |
| Vibrio cholerae | N- | Fucose and | Intestinal | Cholera |
| | methylphenylalanine | mannose | epithelium | |
| | pili | carbohydrate | | |
| Treponema | Peptide in outer | Surface | Mucosal | Syphilis |
| pallidum | membrane | protein (fibronectin) | epithelium | |
| Mycoplasma | Membrane protein | Sialic acid | Respiratory | Pneumonia |
| | | | epithelium | |
| Chlamydia | Unknown | Sialic acid | Conjunctival or | Conjunctivitis |
| | | | urethral | or urethritis |
| | | | epithelium | |

From: http://textbookofbacteriology.net/colonization.html

Further, Table 3 below, indicates suitable parasite and receptor pairings.

TABLE 3
Suitable parasite and receptor pairings
Parasite: receptor

Leishmania: DC-SIGN
Trypanosoma cruzi: LLC-MK2
Malaria: heparan sulfate proteoglycans

A preferred aspect of the invention provides a method of diagnosis of a subject. Specifically, a method of diagnosing the presence or absence of a pathogen in a subject, which method comprises:
(a) obtaining a sample from the subject;
(b) determining the absence or the presence or absence of the pathogen in the sample according to the methods described above;
(c) making a diagnosis based on the results of step (b).

Still further the present invention provides a kit for determining the presence or absence of a pathogen in a sample from a subject, comprising a fusion protein as described above. In particular, the fusion protein comprises a whole or a part of a cell surface receptor and green fluorescent protein (GFP), wherein the cell surface receptor protein or part thereof is one which binds a pathogen during a wild-type infection of the pathogen in vivo. In a particular embodiment the cell surface receptor protein is CD81, CD209 or CD209L.

The kit of the present invention can be used to determine the presence or absence of a pathogen in a sample taken from a subject or in an environmental sample.

Further, the kit may comprise magnetic or inert beads bound to the cell surface receptor protein, e.g. magnetic or inert beads with bound CD81LEL. Alternatively, the kit may comprise a separate cell surface receptor protein, e.g. free CD81LEL, and coupling buffer. A coupling buffer is a buffer with optimal levels of salt, pH, etc. to allow for efficient covalent coupling or hydrogen bonding of peptides/proteins, etc. to inert surfaces or other proteins/peptides/nucleic acids, etc., for example, CD8 to a bead. Examples of suitable coupling buffers include: 0.1 M sodium carbonate, 0.5 M NaCl pH 9.0; 0.1M sodium borate pH 8.5; 0.02M sodium phosphate, 0.2M sodium chloride and 3.0 g/L sodium cyanoborohydride pH 7.5; 0.01M K₂HPO₄, 0.15 M NaCl pH 5.5.

The kit of the present invention may additionally comprise one or more of the following buffers: wash buffer, binding buffer, elution buffer, and diluent buffer.

Wash buffers are used to remove non-specifically bound contaminants and also contain optimal levels of salt and pH, etc. These buffers also often contain non ionic detergents such as Tween-20, nonidet, etc.

Binding buffers optimise the binding of the analyte of interest (e.g. HCV to CD81), such as phosphate buffered saline (PBS).

Blocking buffers block “free” reactive groups thereby preventing contaminants (e.g. secondary antibodies) binding which could artificially increase signal. Blocking buffers may contain for example, bovine serum albumin (BSA), 1M ethanolamine, fish skin gelatine, porcine gelatine, etc.

Elution buffers allow for the analyte of interest to become detached from binding, and contain optimal levels of salt etc, or soluble compounds which directly compete with the bound antigen, e.g. GST fusion proteins bound to a glutathione column will be eluted in a buffer containing 10 mM reduced glutathione. In addition, the pH of these solutions may be raised or lowered compared to the other buffers to optimise elution. Examples include glycine buffers.

Diluent buffers are used to dilute samples to within a working range, and do not interfere with the subsequent reactions. Examples include tris buffers PBS, etc.

Still further, the kit of the present invention may additionally comprise a substrate and a substrate buffer; positive and negative controls; calibrators, detection proteins and amplification proteins.

When the kit comprises receptors coated onto beads, it may find many uses. The receptor-coated beads could, for example, be used in both diagnostics and research. Medical applications could include the clearance of pathogen (e.g. HCV) virions from patient blood in a dialysis-type method. The beads may be mixed with patient samples (blood or biopsies), or with cultured samples. Virus may bind to the beads, which may then be immobilised, for example, on electromagnets. The beads may then be washed to remove contaminants, if desired, for downstream applications. The bound virions may then be disrupted in a small volume of fluid (i.e. concentrated) to release nucleic acids, proteins and lipids, which would then be accessible for further analysis. Alternatively, the GST tag may be cleaved (using established protocols) and purified intact virus attached to CD81 LEL may be used for further analysis in research applications.

### EXAMPLES

### Background and Schematics

FIGS. 3 and 4 provide schematics of how the invention works with the virus (e.g. HCV) and the LEL part of the cell surface receptor protein (e.g. CD81).

The receptor (e.g. CD81 LEL) gene is cloned into a plasmid containing suitable promoter and polyadenylation sequences, and also a purification tag (e.g. GST, polyhistidine, IgGFc, etc.). The plasmid is then transduced or transfected into cells (bacterial or eukaryotic). The resultant expressed receptor protein can be purified using established techniques (e.g. glutathione columns for the GST tag, anti receptor antibody coated columns, or protein A/G columns).

The purified peptide is then coupled to an inert surface such as, but not restricted to; magnetic beads, non-magnetic beads, an immunosorption plate, etc. Plasma containing intact HCV virus is then added, and incubated to allow the virions to contact the immobilised receptor. The plasma is then removed, and the virions re-suspended in a small volume of liquid. This process serves to concentrate the HCV virus, prior to detection either by established means or via the receptor/reporter method outlined above.

The abbreviations used in the following examples have the following meanings:

BSA Bovine serum albumin
CD81 Cluster of differentiation antigen 81
EDAC N-(3-Dimethylaminopropyl)-N′-ethylcarbodiimide hydrochloride
HCV Hepatitis C virus
HCVpp HCV pseudoparticles
HIV Human immune-deficiency virus

### IgG Immunoglobulin G

LEL Large extra cellular loop (of CD81)
mAb Monoclonal antibody
MES 2-(N-morpholino)ethanesulfonic acid
NHS N-hydroxyl succinimide
PBS phosphate buffered saline
PCR Polymerase chain reaction
RT-PCR Reverse transcription PCR
SDS-PAGE sodium dodecyl sulphate polyacrylamide gel electrophoresis

### Example 1

### Using CD81 Coated Beads in an Assay to Concentrate HCV Pseudoparticles

This example details the construction and use of CD81 coated beads in assays to concentrate HCV pseudoparticles (HCVpp), which express the HCV E1E2 on the particle surface.

CD81 is a receptor for HCV binding, The E2 protein on the surface of HCV (as a complex with E1) directly binds to CD81 via a large extra-cellular loop (LEL) (see FIG. 5). In addition, other receptors such as CD209 and CD209L have also been shown to be important in the binding of virus to cells.

### Methods

### CD81LEL-GST Design, Cloning, Expression and Purification

The CD81LEL-GST fusion protein used in this Example was designed using sequences freely available (accession numbers NM004356, EF064749 and BC093047). The amino acid and DNA sequence of the CD81LEL used can be seen in FIG. 6, and below:

SEQ ID 1
FVNKDQIAKDVKQFYDQALQQAVVDDDANNAKAVVKTFHETLDCCGSSTL
TALTTSVLKNNLCPSGSNIISNIFKEDCHQKIDDLFSGK
SEQ ID 2
TTTGTCAACAAGGACCAGATCGCCAAGGATGTGAAGCAGTTCTATGACCA
GGCCCTACAGCAGGCCGTGGTGGATGATGACGCCAACAACGCCAAGGCTG
TGGTGAAGACCTTCCACGAGACGCTTGACTGCTGTGGCTCCAGCACACTG
ACTGCTTTGACCACCTCAGTGCTCAAGAACAATTTGTGTCCCTCGGGCAG
CAACATCATCAGCAACCTCTTCAAGGAGGACTGCCACCAGAAGATCGATG
ACCTCTTCTCCGGGAAG

The design included the CD81LEL previously shown to bind to the E2 envelope protein. The CD81LEL was amplified from a human cDNA library and cloned into the pGEX6p-1 vector (GE Healthcare Life Sciences) which contains a genetically modified GST gene by FusionAntibodies Ltd (Belfast, Northern Ireland) using standard molecular biology techniques. The amino acid and DNA sequences are shown in FIG. 7, and below:

SEQ ID 3
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYAW
PLQGWQATFGGGDHPPKSDLEVLFQGPLGSPEFPGRLERP
SEQ ID 4
ATGTCCCCTATACTAGGTTATTGGAAAATTAAGGGCCTTGTGCAACCCAC
TCGACTTCTTTTGGAATATCTTGAAGAAAAATATGAAGAGCATTTGTATG
AGCGCGATGAAGGTGATAAATGGCGAAACAAAAAGTTTGAATTGGGTTTG
GAGTTTCCCAATCTTCCTTATTATATTGATGGTGATGTTAAATTAACACA
GTCTATGGCCATCATACGTTATATAGCTGACAAGCACAACATGTTGGGTG
GTTGTCCAAAAGAGCGTGCAGAGATTTCAATGCTTGAAGGAGCGGTTTTG
GATATTAGATACGGTGTTTCGAGAATTGCATATAGTAAAGACTTTGAAAC
TCTCAAAGTTGATTTTCTTAGCAAGCTACCTGAAATGCTGAAAATGTTCG
AAGATCGTTTATGTCATAAAACATATTTAAATGGTGATCATGTAACCCAT
CCTGACTTCATGTTGTATGACGCTCTTGATGTTGTTTTATACATGGACCC
AATGTGCCTGGATGGGTTCCCAAAATTAGTTTGTTTTAAAAAACGTATTG
AAGCTATCCCACAAATTGATAAGTACTTGAAATCCAGCAAGTATATAGCA
TGGCCTTTGCAGGGCTGGCAAGCCACGTTTGGTGGTGGCGACCATCCTCC
AAAATCGGATCTGGAAGTTCTGTTCCAGGGGCCCCTGGGATCCCCGGAAT
TCCCGGGTCGACTCGAGCGGCCGC

Expression of the fusion protein was achieved using laboratory strains of E. coli. (BL21 STAR strain). The fusion protein was isolated initially by the isolation of inclusion bodies (a sodium dodecyl sulphate polyacrylamide gel electrophoresis gel (SDS-PAGE) of which can be seen in FIG. 8). Solubilisation of the purified protein and refolding was achieved by dialysis. During refolding, aggregation was not seen. The expressed fusion protein was further purified using a glutathione agarose column (GSTrap column), washed using PBS, eluted using free glutathione (which was subsequently removed by dialysis) and concentrated using column chromatography. The concentration of the fusion protein (0.6 mg/ml) was determined by comparison on a SDS-PAGE gel to known quantities of BSA (FIG. 9) and using a protein assay kit (Micro BCA, Pierce) (FIG. 10). Data for are as follows:

| CD81 | | | | Concentration |
|---|---|---|---|---|
| Dilution | reading 1 | reading 2 | Average | (mg/ml) |
| 1 | 0.2660 | 0.2550 | 0.2605 | 0.62 |
| 2 | 0.1910 | 0.1840 | 0.1875 | 0.32 |
| 4 | 0.1460 | 0.1480 | 0.1470 | 0.15 |

### Conjugation of CD81LEL-GST to Paramagnetic Micro Beads

The CD81LEL-GST fusion protein was covalently linked to Dynal (Invitrogen) 2.8 μm diameter carboxylic acid coated paramagnetic beads. For comparison, biotinylated CD81LEL-GST was mixed with streptavidin coated beads (non-covalent, but nonetheless; strong hydrogen bonding). The concentrations and techniques used were based on the manufacturer's recommendations. The beads were then used in assays to determine if concentration of HCVpp was achievable.

The micro-beads were coated at a concentration of 1 mg of 3% beads to 500 μg fusion protein.

Materials

| | | |
|---|---|---|
| 50 mM MES (Sigma) | 9.76 g/l | Cat # M8250. Lot # 064k5462 |
| pH 6.2 | | |
| 1M Tris-HCl pH 7.4 | 134 g Trizma-hydrochloride (Sigma) | Cat # T3253. Lot # 037k5434 |
| | 18.2 g Trizma Base (Sigma) | Cat # T1503. Lot # 036K5445 |
| | To 1 litre | |
| Wash Buffer pH 7.2 | 0.944 g Disodium hydrogen orthophosphate | Cat # 102495D. Lot # F1485081 |
| | (BDH) | |
| | 0.327 g Sodium-dihydrogen orthophosphate | Cat # 102455S. Lot # A691821 |
| | (BDH) | |
| | 9 g Sodium chloride (BDH) | Cat # 102415k. Lot # K37352833 |
| | To 1 litre | |
| Diluent pH 7.4 | 12.11 g Trizma Base (Sigma) | Cat # T1503. Lot # 096K5404 |
| | 8.77 g Sodium chloride | Cat # 102415k. Lot # K37352833 |
| | 7.44 g EDTA disodium | Lot # 096K0001 |
| | 1.1 g Tween 20 (BDH) | Cat # 083664. Lot # S4799415 |
| | 1 g Nipasept | Lot # GB002774 |
| | 10 ml (1 mg/l) Sarafloxacin | Lot # 709765300 |
| 3% Dynabeads | 2 × 10⁹ beads/ml | Cat # 343.02D. Lot # H2981 |
| M-270 (Invitrogen) | | |
| EDAC (Sigma) | 2.5 mg/ml | Lot # 076K07163 |
| NHS | 1.5 mg/ml | Lot # 537955-476 |
| CD81 fusion protein | | |
| 0.6 mg/ml | | |

Conjugation of CD81LEL-GST Covalently to Carboxylic Acid Activated Beads

0.136 ml Dynabeads (2.72×10⁸ beads) were aliquoted into a 1.5 ml tube. The beads were washed by immobilising using a magnet for 1 minute. The supernatant was removed and 1 ml 50 mM MES was added. This solution was mixed by gentle rotation on a rotamixer for 5 minutes. The beads were immobilised as before and the supernatant removed. The wash step was repeated twice. 0.925 ml 50 mM MES solution was added to the solution free beads. This solution was mixed by rotation for 5 minutes. 25 μl 60 mg/ml NHS dissolved in 50 mM MES was added to the beads along with 25 μl 100 mg/ml EDAC in 50 mM MES to prepare the beads for binding. This solution was rotated for 15 minutes before the supernatant was removed as before. The activation step was repeated twice. 0.9 ml CD81LEL-GST was added to the beads along with 0.5 ml of 50 mM MES to maintain pH. 100 μl 1M Tris-HCl buffer was added to the mixture, which was then rotated for an hour, before the supernatant was removed as before. The beads were gently re-suspended in 1 ml of the wash buffer. The mixture was then rotated for 5 minutes and the supernatant removed as before. The wash step was repeated once and the supernatant removed. The beads were re-suspended in 1 ml diluent buffer and stored at 2-8° C.

The chemistries involved in binding proteins to carboxyl groups are demonstrated in FIG. 11. The dehydrating agent EDAC reacts with carboxyl group on the beads to form an amine-reactive O-acylisourea intermediate. The NHS molecule stabilises the intermediate by converting it to an amine-reactive sulpho-NHS ester which increases the efficiency of EDAC mediated coupling reactions.

### Example 2

### RNA Isolation, Reverse Transcription and PCR Amplification of CD81 Concentrated HCVpp eGFP Gene

### Materials

### HCV Pseudoparticles

MagMAX viral RNA kit—Cat#AM1939 Lot#0709004
Forward primer—Oligo No 70727 Bruce 2F03¾
Reverse primer—Oligo No 70731 Bruce 2A014/4
Autoclaved water
Reverse transcriptase (improm II)—M314A Lot#24139602
Reverse transcriptase buffer×5—Cat# M289A Lot#17198558

### MgCl₂—Cat# A351H Lot#22535642

SYBR green—Lot#125k1212
Agarose—A9539-25G Lot#12kk0157×10 TAE buffer
PCR purification kit—Cat#28104 GR#21166/1 Lot#127147854

### RNase H—Cat#M02975 5000 U/ml Lot#3

RNase X10 buffer—Cat#B0297S Lot#105
Easy A high fidelity PCR master mix Cat#600640-51 Lot#0870448
Quick load 2-log ladder #NO₄₆₉S Lot: 5
Corning thermowell gold PCR tubes 0.5 ml lot#32006023
Corning thermowell gold PCR tubes 0.2 ml lot#08807020

### Autoclaved PBS

### Virus Concentration

100 μl of pseudoparticles (virus concentration 10⁵/ml) and 100 μl coated beads were added to a 500 μl PCR tube. The mix was vortexed at moderate speed for 3 seconds. The processing tube at was incubated at 37° C. for 1 hr. The processing tube was then moved to a magnetic stand to capture beads. The tube was left on the magnetic stand for 30 seconds, followed by careful aspiration and discarding of the supernatant. 100 μl PBS (10 mM) was then added to the sample. The vortexing, incubation, magnetic, and aspiration steps were repeated, and the beads re-suspended in 20 μl of PBS

### MagMax Viral RNA Isolation

To each processing tube was added 200 μl prepared MagMax viral RNA isolation kit Lysis/binding solution, 20 μl of bead mix, 100 μl sample (pseudoparticles) and 75 μl of MagMax viral RNA isolation kit wash solution no. 1. Mixing took place by vortexing for 3 seconds. The processing tube was moved to a magnetic stand to capture beads. The tube was left on the magnetic stand for 30 seconds, followed by careful aspiration and discarding of the supernatant. 112 μl of MagMax viral RNA isolation kit wash solution no. 2 was then added to the sample. The vortexing, incubation, magnetic, and aspiration steps were repeated, and 50 μl elution buffer was added to the sample, and vortexed for 5 seconds. The RNA binding beads were captured on a magnetic stand for 30 seconds. The supernatant, which contains the RNA, was transferred to a nuclease free container,

### Reverse Transcription Protocol

Before PCR can be carried out, the GFP RNA must first be reverse transcribed into cDNA. This was effected using the following protocol:
1. Primers and RNA were added together, melted at 70° C. for 5 minutes then transferred to ice. This step allowed for primer annealing to RNA.
2. The rest of the components were then added after the initial primer/RNA mixture had been chilled on ice.

The reverse transcription components are shown below:

Total RNA=50 μl (50 μl recovered in the purification step)
GFP reverse Primer=5 μl
H₂O=bring up to 100 μl (final volume)=9 μl

### Enzyme Buffer×5=20 μl

### MgCl₂=10 μl

10 mM dNTPs=5 μl
Reverse transcriptase=1 μl

The reverse transcription programme employed was 42° C. for 2 minutes, 95° C. for 5 minutes then hold at 4° C.

### RNase H Treatment

After PCR has finished, 1 μl of RNase H was added in order to digest any remaining RNA strands. This enzyme was added along side 11 μl of X10 RNase buffer. The reaction mixture was placed at 37° C. for 10 minutes then 95° C. for 5 minutes.

### QIAquick PCR Purification Microcentrifuge Protocol

5 volumes of buffer PBI were added to 1 volume of the PCR reaction and mixed. A QIAquick column was placed in a provided 2 ml collection tube. The sample was applied to the QIAquick column and centrifuged for 30-60 s. The flow through was discarded and the QIAquick column was placed back into the same tube. 0.75 ml buffer PE was added to the QIAquick column to wash, and centrifuged for 30-60 s. The flow-through was discarded and the QIAquick column was placed back into the same tube. The column was centrifuged in a 2 ml collection tube (provided) for 1 minute. Each QIAquick column was placed in a clean 1.5 ml microcentrifuge tube. 50 μl Buffer EB (10 mM Tris-Cl, pH 8.5) or water was added to the centre of the QIAquick membrane, and the column centrifuged for 1 minute to elute DNA. For increased DNA concentration, 30 ml elution buffer was added to the centre of the QIAquick membrane, and the column was allowed to stand for 1 minute, and then centrifuged. If the purified DNA is to be analyzed on a gel, 1 volume of Loading Dye is added to 5 volumes of purified DNA. The solution is mixed by pipetting up and down before loading the gel.

Note: All centrifugation steps were at 17,900×g.

### PCR Protocol

The cDNA produced in the above step may now be used and amplified by PCR. This was effected as follows.

### Components:

cDNA=1 μl (some of product from RT-PCR)
GFP Forward Primer=2 μl of a 10 pmole/μl solution
GFP Reverse Primer=2 μl of a 10 pmole/μl solution
H₂O=bring up to 50 μl (final volume)
Easy A high fidelity master mix=25 μl
(Contains dNTPs, enzyme and MgCl₂)

The programme employed involved an initial denaturation step of 95° C. for 2 minutes then 25 cycles of: 95° C. (0.30); 55° C. (0.30); 72° C. (0.10). Then 7.00 at 72° C. followed by holding at 4° C.

### Testing Amplification

A 0.75% agarose gel was prepared using TAE (1 g agarose in 100 ml buffer). DNA was stained with SYBR green (1 μl per 50 μl). 5 μl loading buffer was pipetted to each 50 μl reaction mixture, then 10 μl was pipetted into the wells of the gel. 10 μl of the quick load 2 log ladder with added SYBR green was added to lane one of the gel. It was run at 150 volts, 100 mAmps for 1 hr 30 mins.

### Result

FIG. 12 shows the gel electrophoresis of PCR amplified eGFP gene from HCVpp. The gel comprises: +ve control, HCVpp mixed directly with RNA isolation beads, CD81 coated beads: HCVpp concentrated using CD81LEL-GST covalently coupled to magnetic beads, −ve: negative control (no cDNA template).

FIG. 13 shows fluorescence measurements of the three PCR products. Excitation wavelength: 497 nm, emission reading at 520 nm.

FIG. 14 shows gel electrophoresis of PCR amplified eGFP gene from HCVpp. Key: D: 2 log-DNA ladder, +ve: positive control, HCVpp mixed directly with RNA isolation beads, CD81: HCVpp concentrated using CD81LEL-GST covalently coupled to magnetic beads, CD81B HCVpp concentrated using biotinylated CD81LEL-GST coupled to streptavidin coated magnetic beads −ve: negative control (no cDNA template).

In conclusion, both covalently coupled CD81LEL-GST and biotinylated CD81LEL-GST bound to streptavidin coated magnetic beads can be employed to concentrate HCVpp from a 200 μl starting volume to 20 μl. The HCVpp can thereafter be disrupted for RNA isolation and detection using PCR amplification. Accordingly the effectiveness of methods of detection and/or concentration/purification of pathogens (such as viruses) using cell surface receptors has been demonstrated.

1. A method for determining the presence or absence of a pathogen in a sample, which method comprises:
a) contacting the sample with a whole or a part of a cell surface receptor protein capable of binding the pathogen;
b) allowing the cell surface receptor protein or part thereof to bind the pathogen;
c) determining the presence or absence of the pathogen bound to the receptor protein or part thereof.

2. A method of claim 1, wherein the determining comprises capturing, concentrating, purifying and/or isolating a pathogen in a sample.

3. A method according to claim 1, wherein the method is used with a microfluidic or nanofluidic method.

4. A method according to claim 2, wherein the capturing, concentrating, purifying and/or isolating is carried out as part of a diagnostic flow process.

5. A method according to claim 1, wherein step (a) comprises contacting the sample with only that part of the cell surface receptor protein which binds to the pathogen.

6. A method according to claim 1, wherein the cell surface receptor protein is one which binds to the pathogen during a wild-type infection of the pathogen in vivo.

7. A method according to claim 1, which further comprises the step of concentrating the pathogen bound to the protein.

8. A method according claim 1, which further comprises the step of quantifying the amount of the pathogen in the sample.

9. A method according to claim 1, which further comprises separating the pathogen bound to the protein from the sample.

10. A method according to claim 1 wherein the pathogen is a virus or a bacterium.

11. A method according to claim 10 wherein the virus is an RNA virus.

12. A method according to claim 11 wherein the virus is Hepatitis C virus (HCV).

13. A method according to claim 12 wherein the cell surface receptor protein is selected from CD81 receptor, CD209 receptor and CD209L receptor.

14. A method according to claim 12, wherein the part of the cell surface receptor protein is the large extracellular loop (LEL) of the CD81 receptor.

15. A method according to claim 11 wherein the virus is Human Immunodeficiency Virus (HIV).

16. A method according to claim 15 wherein the cell surface receptor protein is CD4 or CCR5.

17. A method according to claim 11 wherein the virus is Influenza virus.

18. A method according to claim 17 wherein the cell surface receptor protein is a sialoglycoprotein, selected from alpha 2,3-linked sialic acid receptor or the alpha 2,6 linked SA receptor.

19. A method according to claim 11 wherein the virus is rhinovirus.

20. A method according to claim 19 wherein the cell surface receptor protein is ICAM1.

21. A method according to claim 1, wherein the whole or the part of the receptor is conjugated to a solid surface.

22. A method according to claim 21, wherein the solid surface is a bead.

23. A method according to claim 22, wherein the bead is a magnetic bead.

24. A method according to claim 22, wherein the bead is a non-magnetic bead.

25. A method according to claim 1, wherein the whole or the part of the receptor is a fusion protein with a fusion tag.

26. A method according to claim 25, wherein the fusion tag is glutathione-S-transferase (GST).

27. A method according to claim 25, wherein the fusion tag is a fluorescent protein.

28. A method according to claim 1, wherein the presence or absence of a pathogen is determined in, or the capturing concentrating purifying and/or isolating takes place in, a sample from a subject.

29. A method according to claim 28, wherein the sample is a body fluid taken from the subject.

30. A method according to claim 29 wherein the body fluid is selected from blood, urine, serum or plasma.

31. A method according to claim 1, wherein the presence or absence of a pathogen is determined in, or the capturing concentrating purifying and/or isolating takes place in, an environmental sample.

32. A method according to claim 31, wherein the sample is a soil sample, an air sample or a water sample.

33. A method of diagnosing the presence of a pathogen in a subject, which method comprises:
(a) obtaining a sample from the subject;
(b) determining the absence or the presence of the pathogen in the sample by the method of claim 1;
(c) making a diagnosis based on the results of step (b).

34. A method for capturing, concentrating, purifying and/or isolating a pathogen in a sample, which method comprises:
(a) obtaining a sample;
(b) capturing, concentrating, purifying and/or isolating a pathogen in a sample by the method of any one of claim 1.

35. A fusion protein comprising a whole or a part of a cell surface receptor and green fluorescent protein (GFP), wherein the cell surface receptor protein or part thereof is one which binds a pathogen.

36. A fusion protein according to claim 35, wherein the cell surface receptor protein is CD81, CD209 or CD209L.

37. Use of a fusion protein as defined in claim 35 in a method for determining the presence of a pathogen in a sample, or in a method for capturing, concentrating, purifying and/or isolating a pathogen in a sample.

38. Use according to claim 35 in a microfluidic or nanofluidic method, and/or in a diagnostic flow process.

39. Use according to claim 37 comprising
a) contacting the sample with a fusion protein;
b) allowing the fusion protein to bind the pathogen;
c) determining the presence or absence of the pathogen bound to the fusion protein.

40. Use according to claim 39, in a method for diagnosing the presence of a pathogen in a subject.

41. (canceled)

42. A kit for determining the presence of a pathogen in a sample, or for capturing, concentrating, purifying and/or isolating a pathogen in a sample, which kit comprises a fusion protein as defined in claim 35.

43. A kit according to claim 42, wherein the sample is a sample from a subject.

44. A kit according to claim 42, wherein the sample is an environmental sample.


SEQUENCE LISTING (4 sequences)

SEQ ID NO: 1 - protein, 89 aa, Homo sapiens
FVNKDQIAKDVKQFYDQALQQAVVDDDANNAKAVVKTFHETLDCCGSSTLTALTTSVLKNNLCPSGSNIISNLFKEDCHQKIDDLFSGK

SEQ ID NO: 2 - DNA, 267 bp, Homo sapiens
tttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaag

SEQ ID NO: 3 - protein, 240 aa, Artificial Sequence
  Cloning Vector pGEX-6P-1
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYAWPLQGWQATFGGGDHPPKSDLEVLFQGPLGSPEFPGRLERP

SEQ ID NO: 4 - DNA, 724 bp, Artificial Sequence
  Cloning Vector pGEX6p-1
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccggaattcccgggtcgactcgagcggccgc